(19) United States
(12) Patent Application Publication
(10) Pub. No.: US 2004/0019281 A1
(43) Pub. Date: Jan. 29, 2004

(54) Device for the picture providing and spectroscopic diagnosis of tissue

(75) Inventors: Bernd Claus Weber, Karlsruhe (DE); Thomas Goll, Knittlingen (DE); Olaf Schmidt, Vaihingen/Enz (DE); Philipp Eidner, Bretten-Buechig (DE); Stefan Muller, Bretten-Diedelsheim (DE); Nicolas Pereira Delgado, Maulbronn (DE); Lutz Freitag, Hemer (DE)

Correspondence Address: Thomas C. Pontani, Esq., Cohen, Pontani, Lieberman & Pavane, Suite 1210, 551 Fifth Avenue, New York, NY, 10176, US

(73) Assignee: Richard Wolf GmbH

(21) Appl. No.: 10/205,350

(22) Filed: Jul. 25, 2002

(30) Foreign Application Priority Data
Jul. 25, 2002 (DE) 101 36 191.2

(51) Int. Cl. A61B 6/00

(52) U.S. Cl. 600/476

(57) ABSTRACT

The invention relates to a device for the picture-providing and spectroscopic diagnosis of tissue with the alternative or combined use of three diagnosis methods, specifically a mode A for the picture-providing white light diagnosis, a mode B for the picture-providing fluorescence diagnosis and a mode C for the fluorescence-spectroscopic diagnosis. The device comprises a first light means whose light as a beam bundle via a beam path is coupled into an optical fiber leading to an endoscope and a second illumination means whose light as a beam bundle via a second beam path may be coupled into the same fiber-optic. In the first beam path there is arranged an element widening the bundle opening, for the light beam bundle entering into the fiber-optic and in the second beam path there is arranged an element limiting the bundle opening, for the light beam bundle entering into the fiber-optic. Furthermore there are arranged means in the two beam paths with which the light beam bundle may be alternately temporarily released or interrupted. In mode C this permits the specific examination of a selected point wise small tissue region with light from a second beam path with a pseudo-simultaneous, large-surface illumination of the surroundings of the point wise small tissue region with light from the first beam path.

### BACKGROUND OF THE INVENTION

The invention relates to a device for the picture-providing and spectroscopic diagnosis of tissue with the alternative or combined use of three diagnosis methods, specifically a mode A for the picture-providing white light diagnosis, a mode B for the picture-providing fluorescence diagnosis and a mode C for the fluorescence spectroscopic diagnosis, wherein the device comprises an illumination means, whose light as a beam bundle via a beam path is coupled into an fibber-optic fibber leading to an endoscope.

Diagnosis devices of the known type are required for large-surfaced picture-providing white light examination (mode A), for large-surface picture-providing fluorescence diagnosis (mode B) and for point fluorescence spectroscopy (mode C), i.e. for spectroscopic examination of the fluorescence of the smallest of tissue regions (optical biopsy). The spectroscopic examination at the same time relates to the tissue regions which previously have become conspicuous with the picture-providing fluorescence diagnosis or with the picture-providing white light diagnosis.

It is desirable for the examining physician to firstly carry out a usual large-surfaced white light diagnosis (first diagnosis mode A within the context of the present invention), in order firstly under white light to obtain an overview of the tissue to be examined and to set up a prior diagnosis. With this, inasmuch as it is visible under white light, one searches for inflamed, malignant or early-malignant tissue. The examination at the same time is effected by large-surfaced observation of the tissue. For this it must be ensured that the field angle of the object and accordingly also the illumination angle is large enough.

With conventional white light diagnosis however early-malignant tissue cannot be differentiated from benign tissue, which is why it may not be detected. The white light diagnosis has an insufficient sensitivity for this. Accordingly a life-saving or at least significant life-prolonging therapy may not be carried out.

Here the known picture-providing fluorescence diagnosis (mode B within the context of the present invention) represents a decisive advantage. With a suitable object field angle and illumination angle which may be identical to those with white light diagnosis, thus sufficiently large, the physician in the mode of picture-providing fluorescence diagnosis may view the tissue over a large surface, and his attention is quickly drawn to suspect locations by way of intensity and color differences between benign and (early-) malignant tissue.

In the literature it has been shown that with early recognition of cancer by way of additional picture-providing fluorescence diagnosis the sensitivity may be considerably increased with respect to white light diagnosis alone. However deficiencies are still to be observed with the specificity. Thus for example with autofluorescence diagnosis some (early-) malignant lesions are distinguished only by a drop in the fluorescence intensity, the color shifting towards the red spectral region may hardly still be preceived or not at all perceived on account of the fluorescence which under circumstances is greatly reduced with respect to healthy tissue. The suspect location merely appears dark, and colors are hardly to be recognized on account of this. An increase in brightness only leads to the fact that the surrounding healthy tissue acts in an over-irradiated manner and therefore the color changes, indeed at small suspect locations may neither be perceived.

Accordingly, these lesions may hardly be differentiated or not be differentiated at all from healthy tissue which for example alone on account of a changed surface structure (e.g. morphologically heavily structured tissue which acts as a “light trap” and thus lets the healthy tissue region likewise appear dark) likewise leads less fluorescence light to the observer than morphologically normal structured healthy tissue.

Uncertainties on assessing such tissue in all cases render the taking of a sample obligatory in order to detect where possible all potential early-malignant or malignant sources. On account of this the false-positive rate however increases, i.e. the specificity is reduced. To the same extent the time expense and the costs of the examinations increases on account of the greater time expense and additionally required biopsies.

A recording and representation of the fluorescence spectrum (mode C in the context of the present invention) here give a more detailed information on the condition of the tissue concerned by way of drawing up and evaluation and assessment of the spectral composition of the fluorescence emission of the questionable tissue, whilst the previously carried out picture-providing fluorescence diagnosis apart from the information of the changing fluorescence intensity could only give an integral color impression of the tissue concerned as a result of a summing of all spectral components by the eye (taking into account the spectral sensitivity curve of the eye). Details of this are described in DE 196 12 536 A1.

The so-called optical biopsy in the form of a point fluorescence-spectroscopic examination without taking a sample with a subsequent zytological examination thus permits a more accurate and improved assessment and preselection of the tissue than this is possible alone with the large-surfaced picture-providing fluorescence diagnosis.

By way of this preselection therefore less tissue locations need be biopsied and supplied to the pathologist. The evaluation of the fluorescence spectrum and the assessment of the observed point small tissue region, i.e. the evaluation and assessment of the spectral curve, may either be carried out by the physician himself or by a computer connected to the spectrometer, which compares certain points of the emitted fluorescence spectrum with the corresponding points of the previously recorded and stored healthy tissue of the patient to be examined. With this optical biopsy to some extent also edge regions of early-malignant or malignant tissue may be differentiated better or with an increased sureness, from surrounding healthy tissue.

With regard to the fluorescence spectroscopy however the following is to be observed. The spectrum produced for the evaluation and assessment and where appropriate represented on a monitor is the averaged result of the whole (surface) region detected by the spectrometer. The size of the tissue region which leads fluorescence signals to the spectrometer is essentially determined by two components. On the one hand by the fluorescence excitation ray beam which in turn is limited to the top by the numeric aperture of the excitation fiber (fiber bundle), inasmuch as additional optical components are omitted, as well as also by the size of the coupling-in ray beam of the light source into the fiber (fiber bundle), but also on the other hand by the numeric aperture of the detecting fiber (fiber bundle) or by the connecting aperture-limiting devices which may possibly be connected thereto.

If now the local resolution capacity is to be as large as possible when searching for early carcinomas, i.e. if as small as possible lesions are to be recognized and not to be swallowed up in surrounding healthy tissue, the region and/or detected region excited with the fluorescence spectroscopy is to be small as possible. Otherwise when the excited tissue region and the detected tissue region are considerably larger that the suspect location, it may occur that the course of the spectral curve to be evaluated is determined essentially by the healthy tissue surrounding the diseased tissue, since this with regard to the constituent parts delivers a correspondingly larger tissue surface contribution and thus fluorescence contribution for producing the spectral curve. The fluorescence information of the diseased tissue location is then swallowed up in the fluorescence information of the neighbouring healthy tissue, and the diseased location is not recognized as such.

These aspects above all things are to be considered with the spectroscopic autofluorescence diagnosis (mode C), with which the fluorescence of the healthy tissue dominates the fluorescence of the malignant tissue with regard to the intensity. Only with medication-induced fluorescence do these considerations play less of a part, since here in the ideal case only the early-malignant or malignant tissue is enriched with the fluorescing medication, and the healthy tissue surrounding the diseased tissue fluoresces relatively weakly or not at all. With medication-induced fluorescence the situation becomes more critical when for example remitted excitation light is detected for an increase in the contrast.

In total this means that for conventional white light diagnosis as well as for picture-providing fluorescence diagnosis one desires distally large illumination or excitation ray beams and to the same extent large object field angles of the picture channel. With the point fluorescence spectroscopy in contrast (mode C) the excitation ray beam at the distal end of the endoscope or the numeric aperture of the detection fiber or an aperture-manipulatable device possibly additionally attached to the spectrometer must be able to be set adequately small corresponding to the desired location resolution capacity.

If however this tissue region from which fluorescence signals are to be led to the spectrometer is also to be made visible to the examining physician, which in the case of autofluorescence in contrast to medication-induced fluorescence is of great importance for the above mentioned reasons, then this may be effected only by a small excitation light spot which corresponds to this tissue region and which then e.g. In color is set apart from the surrounding, conventionally white illuminated tissue. For example the excitation light spot as a “blue excitation point” on the suspect tissue may be differentiated from the surrounded white-illuminated tissue. The physician thus by way of this and only this may ascertain from which region or part region of the tissue area which is illuminated white is formed.

A system which permits the application of all three mentioned diagnosis methods is known from DE 196 18 963 C2. The conventional white light examination (mode A) and the picture-providing fluorescence diagnosis (mode B) are effected with a first construction with regard to apparatus. In order to be able to operate still further point wise fluorescence spectroscopy (Mode C) in DE 196 18 963 C2 there is suggested an auxiliary device which is designed as an attachment for the endoscope. The auxiliary device contains a beam splitter which ensures that not more than the total light made available by the endoscope picture guide is led to the observing eye or to the connected camera, but only a part of this light is coupled out in order to be led to a spectrometer.

This procedure has the following disadvantages. The auxiliary device attached onto the endoscope increases the weight as well as the geometric dimensions of the equipped diagnosis instrument and by way of this compromises is handling ability. For this reason for a comfortable handling whenever possible one must do away with elements fixed on the endoscope.

With the known state of the art the following condition is also of a problem. For autofluorescence diagnosis it is generally the case that the low intensity of the autofluorescence light almost always represents a problem, which inasmuch as one may omit cumbersome and thus disadvantageous image intensifiers, mostly results in the necessity of the integration of several video frames. This in turn leads to a degrading of the quality when reproducing the picture, which is manifested in the reduced picture repetition rate. If finally as in the case of the previously mentioned apparatus, autofluorescence light is further decoupled, in order to lead this constituent part to the spectrometer, the problem of the low quantity of autofluorescence light in the direct view or camera channel is aggravated even more, i.e., the picture quality with the picture-providing fluorescence diagnosis method which is already compromised is worsened even more by the picture integration which is now worsened to an even greater extent.

With the auxiliary device according to DE 196 18 963 C2 the diameter of the so-called central spectral detection field, thus the diameter of that tissue region whose fluorescence is led to the spectrometer and which thus forms the basis for the produced spectrum is determined by the focal width of the lens in the auxiliary device on the one hand and by the diameter of the connected fiber or fiber bundle on the other hand. Accordingly a change of this diameter of the spectral detection field may not be realized without expense and practically may not be made during an examination of a patient, at least not when there is to exist the possibility of being able to carry out frequent adjustments.

The fluorescence spectroscopy is effected with the solution according to DE 196 18 963 C2 simultaneously to the picture-providing fluorescence diagnosis, since it is indeed just during the picture-providing fluorescence method that a part of the fluorescence light is decoupled. The picture which is delivered by the picture-providing fluorescence diagnosis serves for localizing or locating the location of the fluorescence spectroscopy location or the region in which this spectroscopy location is located. The spectral detection field is located centrally, thus in the middle of picture delivered by the picture-providing fluorescence diagnosis. With autofluorescence diagnosis however as already shown, the fluorescence signals are relatively weak, and on account of the necessity of the integration of several video frames which results from this with the application of a camera, the picture quality on the monitor is in no way comparable to that of white light diagnosis. For this reason it is advantageous when carrying out the fluorescence spectroscopy to orientate oneself on the white light picture instead of on the picture which is delivered by the picture-providing fluorescence spectroscopy. The tissue region which surrounds the potential lesion, with which the point fluorescence spectroscopy is carried out, for the best possible orientation of the examining physician, should be represented in the usual good white light picture quality. This is not the case with the device known from DE 196 18 963 C2.

A further disadvantage with the known device is the fact that the examining physician indeed is not informed of the exact location and above all not on the exact size of the tissue location (diameter) which delivers the information for point wise fluorescence spectroscopy. The spectroscopically examined location although lying in the centre of the picture to be seen at the ocular view on account of the arrangement of the optics, which the physician knowns, he however does not explicitly see this. This centre is however not always easy to determine on account of optical illusions which may results from the picture content. The same applies to the size and the diameter of the spectroscopically evaluated tissue location. With regard to this the situation becomes even more confusing when the above described measures are carried out for changing the diameter.

Another system is known from the book “bronchoscopy” U.B.S. Prakash, Mayo Foundation, Raven Press Ltd., New York, Chapter 15. Here, as with the previously described invention there is also realized the idea of the fluorescence diagnosis with a pseudo-simultaneous white light diagnosis. For this there is provided a chopper wheel with which a circular segment contains a fluorescence excitation filter. This ensures that for a fraction of the rotary time of the chopper wheel the tissue is excited with the correspondingly filtered light. In the same time interval—and only in this—the signal emitted by the tissue is led to a fluorescence detector. A band width filter in the chopper wheel ensures that the excitation light remitted from the tissue is filtered out and only the fluorescence light may pass. During a further fraction of the rotary time of the chopper wheel there is effected an illumination and observation of the tissue under unfiltered illumination light (white light), i.e. no fluorescence excitation filter is located in the illumination beam path. The input of the fluorescence detector is now blocked by the chopper wheel and accordingly obtains no light. This procedure with a chopper wheel and thus the realized “time-sharing-method” permits a fluorescence diagnosis of picture-providing or spectral-analytic type with a pseudo-simultaneous observation of the tissue to be examined under unfiltered illumination light (white light) as an orientation aid or for localizing the tissue region examined with the fluorescence method.

However this device too is not suitable for the point fluorescence spectroscopy with a high local resolution and with display, i.e. optical highlighting of those tissue regions which supply signals to the spectrometer, with the pseudo-simultaneous large-surfaced surrounding illumination as an orientation aid and localizing the point wise spectroscopically examined tissue region. With this device, as in DE 196 18 963 C2, there exists the problem that under the precondition that for reasons of compactness and comfortable handling only one light projector with one light connection is to be used, only one fiber/fiber bundle is to be used which must serve for illumination and for fluorescence excitation. The fluorescence excitation cone of light and the illumination cone of light are accordingly the same size. If the tissue is to be able to be viewed over a large surface and in spite of this a point fluorescence spectroscopy is to be carried out, the setting of the local resolution, i.e. the setting of the size of the detected tissue region must be carried out via the detection fiber/detection fiber bundle, e.g. In the manner as is described in DE 196 18 963 C2. Accordingly however the location and diameter of the detected tissue region may not be optically highlighted by the examining physician from the larger-surfaced tissue surrounding the suspect location and irradiated with illumination light, and thus may not be made visible.

The system known from “bronchoscopy” was also developed for medication-induced fluorescence diagnosis, with which—as already mentioned—with a suitable manner of proceeding one does away with the display of the detected tissue region, since with medication-induced fluorescence in any case only diseased tissue may be fluoresced.

### BRIEF SUMMARY OF THE INVENTION

Accordingly it is the object of the present invention to provide a diagnosis device which overcomes the previously mentioned disadvantages. Furthermore a quick and simple switch-over between the individual modes should be possible. The device should also be designed compact, and the three diagnosis methods should be able to be carried out with only one light projector which contains two illumination means, with one light connection, i.e. one fiber-optic exit.

This solution is achieved by the device specified in claim 1. Further advantageous features of the device are specified in the dependent claims.

The device contains two illumination means. The beam path of the second illumination means before leaving the device is superimposed on the beam path of the first illumination means. The light beam bundle of both is then coupled into a fiber-optic connected to an endoscope. In the first beam path there are arranged elements which have the effect that the light of the first illumination means with a relatively large bundle opening are led into the fiber-optic. In the second beam path there are arranged elements which have the effect that the light of the second illumination means is introduced into the fiber-optic with a comparatively small bundle opening.

In the first beam path there are arranged means which temporarily release or interrupt the light beam bundle of the first beam path.

The guiding of the light of the second beam path to the fiber-optic is effected by way of mirrors of which at least one must be part-transparent. Behind the part-transparent mirror there may be arranged a spectrometer. At the same time it is advantageously envisaged that in front of the spectrometer there are provided means with which the light beam bundle which is led from the examined tissue to the spectrometer, is temporarily released or interrupted. These means may also be arranged such that they may simultaneously release or interrupt the light beam bundle of the second beam path from the second illumination means to the fiber-optic.

The means for the temporary release or interruption of the light beam bundle consist of a first and a second chopper wheel. These may in each case have an opaque disk which in each case have a recess over a defined angular region.

The angular region of the recesses of the two synchronously drivable chopper wheels are formed complementarily to one another such that a covering or opaque region of the second chopper wheel corresponds to the removed region of the first chopper wheel and that a removed region of the second chopper wheel corresponds to the covering or opaque region of the first chopper wheel.

As an element for the bundle opening widening at the location of the coupling of the light of the first beam path into the fiber-optic, one considered a comparatively heavily focusing optical element such as e.g. a lens of a short focal length. The same effect with regard to the bundle opening may however also be achieved with an oblique coupling of the light beam bundle out of the first beam path relative to the optical axis of the fiber-optic or from a combination of both measures.

A telescope with a suitable focal length ratio of its two lenses and/or an aperture-limiting diaphragm in the collimated second beam path may be considered as an element for limiting the bundle opening at the location of the coupling of the light of the second beam path into the fiber-optic, wherein the aperture-limiting diaphragm may exist by way of the limited extension or mounting of one or more optical elements in the second beam path. The same effect with regard to the bundle opening may however be achieved in that as a second illumination means one uses a light source which emits a collimated light beam bundle with a small diameter.

As a fiber-optic there may be provided a fluid fiber-optic or a fiber bundle passing through up to the distal end of the endoscope or an individual fiber passing through up to the distal end of the endoscope, wherein ideally fiber-optics with a high transmission in the fluorescence excitation bandwidth are to be used.

The second illumination means which provides the light of the second beam path is ideally a compact laser, e.g. a diode laser, ideally with a collimated light beam bundle of a small diameter and an emission range which lies in the fluorescence excitation band width of the tissue to be examined. However a light diode or a light diode array with a suitable emission range and preassembled beam-forming optics or a mixed gas lamp, a short arc lamp or incandescent lamp with a preassembled optical bandwidth filter and beam-forming optics are also conceivable.

The point wise fluorescence excitation, the fluorescence detection and the illumination of the healthy tissue surrounding the suspect location with white light are effected in the operating mode C of the point wise fluorescence spectroscopy via the same fiber-optic, specifically that which is also used in the two other operating modes A and B for the illumination and excitation respectively. This means that no additional fibers or fiber bundles are required for the point fluorescence spectroscopy. The instrument channel of the endoscope remains permanently free, for example for taking samples, and the handling of the system may be effected in a simple way and manner.

A simple concept results for the construction of the endoscope, since only one fiber-optic is used, specifically that which is used in the operating modes A and B for illumination and excitation, for the illumination or excitation as well as for the spectrometer detection. The usual optics of an endoscope are sufficient. The construction with regard to apparatus is thus kept simple in accordance with the object of the invention.

The change in the diameter of the so-called central spectral detection field, thus of the diameter of that tissue region whose fluorescence is led to the spectrometer, may be accomplished simply by adjusting an element such as for example an iris diaphragm or a diaphragm wheel or likewise having several diaphragms with a different diameter, in the light source in the excitation channel for the fluorescence spectroscopy, i.e. in the second beam path.

Furthermore a quick and simple switching to and fro between the diagnosis methods of the individual modi A, B and C permits the location of the suspect locations in the conventional white light mode (mode A) or in the large-surfaced picture-providing fluorescence diagnosis mode (mode B) and a subsequent quick switch-over into the mode of the point wise fluorescence spectroscopy. By way of this, with the so-called optical biopsy a more detailed evaluation by way of the fluorescence spectrum is made possible.

With the device according to the invention it is possible to carry out all three mentioned diagnosis methods with one light projector with one light connection, i.e. one fiber-optic exit, and thus with one optical connection of the light source and endoscope.

In addition with bundle openings of different sizes one couples into the same fiber-optic for all diagnosis modes, and specifically white light in the modes A and C and the excitation light in the mode B, from the first beam path with a large bundle opening at the location of the coupling-in into the fiber-optic, and the excitation light in mode C for the point excitation, from the second beam path with a small bundle opening at the location of the coupling-in into the fiber-optic—the proximal-side coupling-in of light with a large bundle opening with the described construction distally effects a large-surfaced illumination or fluorescence excitation; a proximal-side coupling-in of light with a small bundle opening on the other hand with the explained construction distally produces a small-surface or even point illumination or fluorescence excitation. By way of this in mode C it is possible to produce a large-surface white background illumination for orientation for the examining physician (large illumination light cone at the distal end of the illumination or excitation window of the endoscope), and to superimpose the tissue region which delivers the fluorescence light led to the spectrometer, as a suitably small and in the case of the autofluorescence excitation blue spot, onto the white light (smaller or more slimline excitation light cone at the distal end of the illumination or excitation window of the endoscope) and thus of making the location and diameter of the spectrometrically examined region directly visible to the user.

The suggested device is also characterized by a very compact construction. All three examining modes may specifically be carried out with only one light projector. Furthermore the device in the diagnosis mode of the point fluorescence spectroscopy permits the examination on a conventional large-surface white light picture. The location and the diameter of the dimensions of the point wise spectroscopically examined suspect tissue region at the same time may be clearly recognized and differentiated from surrounding tissue which is illuminated white merely for an improved orientation and localizing. The point spectroscopically examined suspect tissue region, thus that whose fluorescence light has been used for producing the spectrometer curve, is highlighted significantly as a blue spot from the large-surfaced surrounding tissue which is not suspect and which remits white illumination light.

Above all there exists the possibility of a simple diameter change of the point spectroscopically examined tissue region and thus the possibility of size adaptation of the spectroscopically detected location to the size of the suspect location. At the same time the diameter change, thus the changed size of the spectroscopically examined tissue region, is directly visible, specifically as the size change of the “blue excitation light spot” against the background of the white illuminated tissue.

Finally there results a simple handling of the device on changing between the individual modes of diagnosis. The change between the different methods of diagnosis requires only the actuation of a switch, a button or likewise. It is not necessary to reinsert fiber-optics, to place an auxiliary device onto the endoscope or even to change the endoscope.

### BRIEF DESCRIPTION OF THE DRAWINGS

In the drawings there is schematically shown one embodiment example of the invention. There are shown in:

FIG. 1 the construction of a diagnosis device,

FIG. 2 the plan view of the disk of a first chopper wheel,

FIG. 3 the plan view of the disk of a second chopper wheel and

FIG. 4 the plan view of the disk with diaphragms having a different diameter.

### DETAILED DESCRIPTION OF THE INVENTION

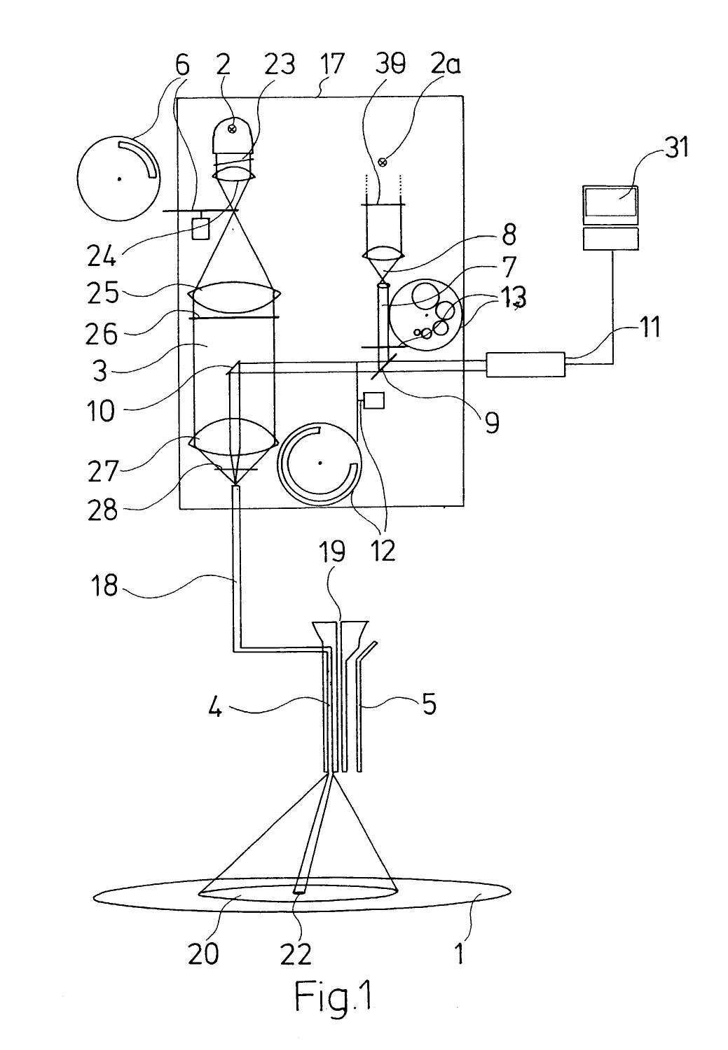

The diagnosis device in FIG. 1 consists of a light projector 17 which is boxed in by the drawn-in rectangle. The light projector 17 has a first illumination means 2 which emits the focused light into a first beam path 3. Via a fiber-optic 18 the light goes from the first beam path 3 into the illumination or excitation fiber 4 of an endoscope 5.

The distal end of the endoscope 5 is directed to the tissue 1 to be examined. The tissue may be observed via an ocular 19 which is not shown in detail. The optics in the first beam path, for example the lens 27 of a comparatively short focal length, as well as the further light guiding via the fiber-optic 18 and the illumination or excitation fiber 4 have the effect that the light which is led from the first illumination means 2 via the first beam path 3 up to the tissue 1, here illuminates a relatively large region 20. The examining physician with the ocular 19 may view and assess over a large surface a relatively large tissue region 20 by way of the white light originating from the first illumination means 2.

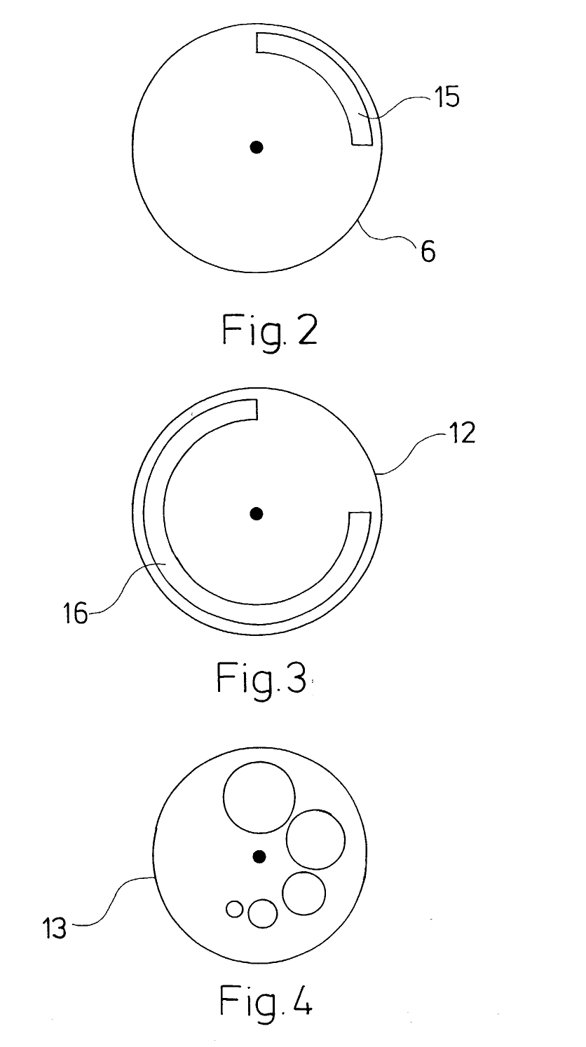

In this examining mode A of the picture-providing white light diagnosis the illumination means 2 supplies light to the tissue 1 permanently and without interruption. A first chopper wheel 6 located in the first beam path 2 is designed such that in a controllable manner it either blocks or releases the light beam bundle from the illumination means 2 to the tissue 1. For this reason the chopper wheel in mode A is stationarily rotated or positioned such that a recess 15 in the chopper wheel disk 14 (FIG. 2) ensures a permanent passage of light.

In the light source 17 there is located a second illumination means 2a which emits a collimated light beam bundle into a second beam path 7. This firstly runs parallel to the first beam path 3. Here there may be arranged a lens system 8 (telescope) consisting of two elements, wherein in place of the telescope 8 one may provide any other element reducing the diameter of the beam, for example an aperture-limiting diaphragm. The aperture-limiting effect may also be achieved by a suitably limited extension of at least one element located in the beam path. It is only important for the size of the diameter of the parallel light beam bundle in the second beam path 7 at the location where it transmits the lens 27 to be relatively small. By way of this it is ensured that the light beam bundle which leaves the endoscope 5 and which originates from the second beam path 7 impinges on the tissue with a comparatively small diameter 22. If one may form the size of the diameter of the collimated light beam bundle in the second beam path 7 and thus the size of the excitation light cone leaving the endoscope such that it can be adjusted, then this may for example be effected via an iris diaphragm or via an adjustable diaphragm wheel 13 comprising several diaphragms with different diameters.

The light beam bundle of the second beam path 7 reduced in diameter via a part-transparent mirror 9 and a mirror 10 is superimposed with the second beam path 3 and led to the endoscope 5 via the fiber-optic 18. Via the illumination and excitation fiber 4 the light reaches the tissue 1, but however on account of the reduced diameter of the collimated light beam bundle at the location of the transmission from the lens 27 and thus on account of the reduced bundle opening of light beam bundle from the second beam path 7 at the location of the coupling into the fiber-optic 18, it reaches only onto a considerably smaller region 22 than the light from the first beam path which illuminates the larger tissue region 20. The part-transparent mirror 9 is designed such that it transmits excitation light and remits fluorescence light.

Behind the part-transparent mirror 9 there is located a spectrometer 11 which at least temporarily is in releasing optical connection and into which fluorescence light emitted by the tissue region 22 may reach for the purpose of spectral analysis. The result of the spectral analysis may be shown on a monitor 31 as a spectrometer curve.

For the point fluorescence spectroscopy (mode C) one must ensure that only and exclusively fluorescence light originating from the tissue 1 from the small excited region 22 gets to the spectrometer 11, but no white light of the first illumination means 2 which is remitted by the tissue. For this a second chopper wheel 12 is arranged in front of the spectrometer 11 which is constructed analogously to the chopper wheel 6. The disk of this chopper wheel 12 (FIG. 3) has a recess 16 which extends over a defined circumferential region and is complementary to the recess 15 of the disk of the first chopper wheel 6 inasmuch as with a simultaneous rotation of both chopper wheels 6 and 12 light from the first illumination means 2 which via the first beam path 3 gets to the tissue to be examined and from this is remitted and which via the fiber 4, the fiber-optic 18 as well as the mirror 10 which is not part-transparent gets to the location of the chopper wheel 12, can never get into the spectrometer 11. By way of the positioning of the chopper wheel 12 in front of the part-transparent mirror, it is achieved that for example in mode A when the spectrometer is blocked no blue spot produced by the illumination means 2a is superimposed with the white illumination of the object field.

In FIG. 1 apart from the chopper wheels 6 and 12 their disks are shown in a plan view from which one may deduce the cooperation of the recesses 15 and 16 with regard to the position. In a corresponding manner a plan view of a diaphragm wheel 13 having several diaphragms with different diameters is also sketched (see also FIG. 4).

In FIG. 1 there are drawn in further details which comprises the light source 17. The first light means 2 is a white light source, wherein an arc lamp with a mirror reflector (paraboloid or ellipsoid) is preferably used. However a condenser system is also conceivable. A spiral wound filament lamp (e.g. a halogen lamp) may also be considered. A filter 23 acts as a bandpass filter which filters out IR an UV radiation. This is effected partly also already by the reflector coating of the illumination means 2. The lens 24 produces a first focus in which the chopper wheel 6 ideally but not necessarily is located. If an elliptical reflector is used, one may omit the lens 24 and the chopper wheel 6 is ideally positioned in the second focal point of the ellipsoid.

The chopper wheel 6 with the conventional white light diagnosis (mode A) and with the picture-providing fluorescence diagnosis (mode B) is always located in the rest position and on let-through (the recess 15 is in the beam path). The lens 25 produces a collimated beam path into whose course there is pivoted a filter 26 with the picture-producing fluorescence diagnosis, whose transmission properties are matched to the optimal fluorescence excitation of the biological tissue to be examined, such as something like a blue filter with the autofluorescence excitation of human tissue for example in the bronchial tract or esophagus.

The filter 26 thus from the broad bandwidth white light of the illumination means 2 selects the required optimal spectral range for the fluorescence excitation. With conventional white light diagnosis this filter is pivoted out of the collimated beam path. The lens 27 focuses the blue light in the mode B of the fluorescence diagnosis or the white light illumination in mode A of the conventional white light diagnosis and in mode C of the point fluorescence spectroscopy so greatly that at the distal end of the endoscope 5 connected to the light source 17 via the fiber-optic 18, the excitation or illumination ray beam is sufficiently large, sufficiently large in the context that with a suitable distance between the endoscope tip and the tissue a sufficiently large tissue region 20 is illuminated and may be seen with a good overview.

The fluorescence excitation light with the picture-providing fluorescence diagnosis (mode B) or the white illumination light with the conventional white light diagnosis (mode A) and the white surrounding illumination with the point wise fluorescence spectroscopy (mode C) are coupled into the fiber-optic 18. A diaphragm 28 which does not limit the aperture permits a control of the light flux quantity led up to the tissue.

In the operating mode C of the point fluorescence spectroscopy the chopper wheel 6 begins to rotate at a high frequency such as for example at the video frequency. The filter 26 for the fluorescence excitation in this mode is pivoted out of the beam path. If the chopper wheel 6 is in the position “let-through” the white light reaches the fiber-optic 18 for the fraction of the revolution duration corresponding to the size of the open circular segment 15, for the remaining fraction of the revolution duration, during which the chopper wheel 6 stands in the beam path in a blocking manner, no white light reaches the fiber-optic and thus the tissue to be examined. Instead of this now light of the illumination means 2a is coupled into the fiber-optic 18 via the beam path 7 and via the mirrors 9 and 10, as well as via the lens 27.

A filter 30 which is permanently located in the beam path 7 selects the excitation light ideal for the fluorescence spectroscopy from the light of the illumination means 2a. If with 2a it is already the case of an illumination means with a spectrally relatively narrow emission band, such as a laser, and this emission band lies completely in the fluorescence excitation band, then one may omit the filter 30.

The fluorescence spectroscopy should, as explained above, be effected advantageously point wise, i.e. as small as possible excitation light cone 22 at the distal end of the illumination or excitation window 4 should permit correspondingly high-defined fluorescence spectroscopy with regard to the location and thus the inclusion and assessment of correspondingly small lesions. The diameter of the collimated beam bundle from the beam path 7 at the location of the lens 27 must be correspondingly small, i.e. the reduction of the diameter of the collimated beam bundle of the illumination means 2a must be correspondingly high. This is effected via the selection of the focal length ratio of the lenses of the telescope 8: the larger the quotient of the focal width of the lens proximal to the illumination means 2a divided by the focal width of the lens of the telescope 8 which is distant to the illumination means 2a, the greater is the reduction in the diameter of the collimated beam bundle emitted by the illumination means 2a. A limited diameter of the mirror 10 for example has the effect of reducing the beam diameter to the same extent. If the illumination means 2a consists of a laser for example which emits a collimated beam with a suitably small diameter, then under circumstances one may omit further measures reducing the diameter of the beam. A further device which acts in an optically damping manner and is not shown, such as for example a neutral filter may permit a regulation of the intensity of the excitation light producing the fluorescence.

In a further embodiment form an adjustable diaphragm wheel 13 (FIG. 4) comprising several diaphragms with different diameters is brought into the beam path, wherein also other aperture-adjustable devices such as for example an iris diaphragm are conceivable. In this embodiment form the focal width ratio of the lenses of the telescope 8 may lie close to one or one may completely omit the telescope 8.

By way of rotating the diaphragm wheel 12 the excitation light cone and thus the (local) resolution capacity is almost infinitely adjustable with fluorescence spectroscopy. If the suspect location is large-surfaced, a large diaphragm in the diaphragm wheel 13 is selected in order to excite almost the whole suspect tissue surface. In the case that a higher resolution capacity is demanded, because the suspect tissue region only has a relatively small diameter, one may select a small diaphragm in the diaphragm wheel. With this it is ensured that the course of the determined spectral curves in the case of autofluorescence is not determined or co-determined by the fluorescence of the tissue neighbouring the suspect location.

The second chopper wheel 12 in the diagnosis mode C of the point wise fluorescence spectroscopy rotates between the mirrors 9 and 10 synchronously, i.e. with a same (comparatively high) rotational frequency and in a fixed phase to the movement of the chopper wheel 5. The size of the recesses 15 and 16 (FIGS. 2 and 3) is only one example for their design. If however the recess 15 is fixed with one chopper wheel 6, the other recess 16 on the other chopper wheel 12 results automatically. During the illumination of the tissue with white light, the first chopper wheel 6 just for this moment is located in let-through, the entry to the spectrometer 11 is covered; furthermore fluorescence excitation light of the illumination means 2a cannot reach the tissue 1. In the excitation phase of the tissue however the chopper wheel 6 now blocks, the entry of the spectrometer 11 is uncovered. By way of the high frequency of the two chopper wheels, for example video frequency, the point wise fluorescence excitation and the illumination of the tissue surrounding the suspect location with white light appears quasi simultaneously. Furthermore in front of the spectrometer 11 one may arrange a filter (not drawn in FIG. 1) which only transmits the fluorescence light, but blocks light outside this spectral region. This job may already be assumed by a mirror 9 when this is designed as a suitable interference filter with suitably strict specifications. The spectrometer 11 may thus only receive fluorescence light, but never white illumination or excitation light remitted by the tissue.

The system contains a central control unit which with switch-over procedures between the individual examination modes coordinates subsequent courses in the light source 17 and on the spectrometer 11. If the device is switched into the mode A of conventional and therefore large-surfaced white light diagnosis, the chopper wheel 6 is braked (inasmuch as it was previously rotating) and during this operating mode remains in the rest position, and specifically in the let-through position with respect to the light of the first beam path 3. Simultaneously one ensures that the filter 26 is pivoted away. The total white light is coupled into the fiber-optic 18. The second chopper wheel 12 which is positioned between the mirrors 9 and 10, inasmuch as it was previously in rotational movement, is likewise braked and during the whole time in this operating mode remains blocking in the rest position. By way of this, on the one hand it is prevented that illumination light remitted by the tissue gets into the spectrometer and on the other hand it is avoided that in the mode A an undesired blue spot from the illumination means 2a appears on the tissue.

When switching into mode B of the picture-providing flourescence diagnosis the positions of the chopper wheels remain unchanged, i.e. the chopper wheel 8 remains resting and in let-through, and the chopper wheel 12 likewise remains still and is blocking. Simultaneously the filter 26 is pivoted into the beam path 3.

If one switches into the operating mode C of the point wise fluorescence spectroscopy the central control unit ensures that the filter 26 is pivoted out of the beam path 3, by which means white light may be coupled into the fiber-optic 18. Both chopper wheels 6 and 12 start to rotate at a high frequency, for example at the video frequency and specifically in a manner such that in the pass-through position of the chopper wheel 6 the chopper wheel 12 which is positioned between the mirrors 9 and 10 blocks. In the phase of the white light illumination of the tissue 1 thus the spectrometer receives no light and the tissue is also not excited point wise with light from the beam path 7. In the blocking position of the chopper wheel 6 the chopper wheel 12 between the mirrors 9 and 10 is transmitting, i.e. the tissue 1 is excited in a point wise manner with the light from the light source 2a filtered via the filter 30 (is as much as the type of illumination means 2a demands this) and which is coupled into the fiber-optic via mirrors 9 and 10 as well as the lens 27, and the spectrometer may receive fluorescence light which is led via the endoscope 5, the fiber-optic 18 and the mirror 10 and transmitted through the semi-transparent mirror 9.

Whilst in FIG. 1 it is shown how the diameter of the light beam bundle in the second beam path may be reduced to the desired small value by way of a telescope, this reduction in diameter may also be effected in any other way by way of a suitable bundling or limiting element.

A particularly simple design with regard to this results when it is envisaged that whilst omitting the telescope 8 one applies a mirror which is kept suitably small in diameter. On account of the oblique position of 45 degrees an elliptical mirror 10 is then applied so that its projection surface in the direction of the optical axis of the first beam path 3 becomes circular. At the same time there results also the advantage that in mode A or in mode B only a small part of the light of the first illumination means 2 at the rear side of the mirror 3 which is preferably assembled rigidly in the beam path 3 is blocked. A light loss in the modes A and B is thus almost completely prevented. On however may also envisage designing the mirror 10 such that it may be folded out of the beam path 3 in order not to have any light losses in the diagnosis modes A and B.

The mirror 9 is designed such that it is high reflecting only to the light exciting the fluorescence, e.g. blue light with the autofluorescence diagnosis. On the other hand it is designed such that it acts in a highly transmitting manner for the fluorescence light. The fluorescence light behind the mirror 9 is coupled into the spectrometer 11 either directly or via a fiber/fiber bundle which is located in the light projector and thus does not hinder the handling of the system.

The spectrometer may, as is shown in FIG. 1, be positioned outside the light source or may be accommodated in the light source housing so that the whole system becomes even more compact.

1. a device for the picture-providing and spectroscopic diagnosis of tissue with the alternative or combined use of three diagnosis methods, specifically a mode a for the picture-providing white light diagnosis, a mode b for the picture-providing fluorescence diagnosis and a mode C for the fluorescence-spectroscopic diagnosis, wherein the device comprises an illumination means, whose light as a beam bundle via a beam path is coupled into a fiber-optic leading to an endoscope, wherein the device apart from the previously mentioned first illumination means comprises a second illumination means whose light as a beam bundle via a further second beam path before leaving the device is superimposed with the other first beam path and then coupled into the fiber-optic, wherein in the first beam path there is arranged a first element widening the bundle opening for the light beam bundle of the first beam path entering the fiber-optic and wherein in the second beam path there is arranged an element limiting the bundle opening for the light beam bundle of the second beam path entering the fiber-optic.

2. A device according to claim 1, wherein in the first beam path there are arranged means with which the light beam bundle may be partly released or blocked.

3. A device according to claim 1, wherein the guiding of the light of the second beam path to the fiber-optic is effected by way of a part-transparent mirror.

4. A device according to one of the claims 1 to 3, wherein behind the mirror there is arranged a spectrometer and wherein in front of the spectrometer there are arranged means with which the light beam bundle in front of the spectrometer may be temporarily released or interrupted.

5. A device according to one of the claims 1 to 4, wherein the means for the temporary release and interruption of the light beam bundle consists of a first chopper wheel and second chopper wheel which may be driven synchronously, and wherein both chopper wheels have opaque surfaces which over a region defined in each case comprise recesses.

6. A device according to claim 5, wherein the recesses of the two chopper wheels are formed complementarily to one another such that the removed region of the first chopper wheel corresponds to a covering or opaque region of the second chopper wheel and wherein a covering or opaque region of the first chopper wheel corresponds to a removed region of the second chopper wheel.

7. A device according to one of the claims 1 to 6, wherein the element widening the bundle opening, for the light beam bundle entering into the fiber-optic from the first beam path consists of a strongly focusing optical element, for example the lens with a short focal length.

8. A device according to one of the claims 1 to 6, wherein the element widening the bundle opening, for the light beam bundle entering into the fiber-optic from the first beam path is realized with an oblique coupling of the light beam bundle from the first beam path into the fiber-optic.

9. A device according to one of the claims 1 to 6, wherein the element widening the bundle opening, for the light beam bundle entering into the fiber-optic from the first beam path consists of an element of claims 7 and 8 alone or of a combination of these elements.

10. A device according to one of the claims 1 to 6, wherein the element limiting the bundle opening, for the light beam bundle entering into the fiber-optic from the second beam path consists of a illumination means which emits a parallel light beam bundle with a small diameter.

11. A device according to one of the claims 1 to 6, wherein the element limiting the bundle opening, for the light beam bundle entering into the fiber-optic from the second beam path consists of a telescope with a suitable focal length ratio of its two lenses.

12. A device according to one of the claims 1 to 6, wherein the element limiting the bundle opening, for the light beam bundle entering into the fiber-optic from the second beam path consists of an aperture-limiting diaphragm in the second beam path.

13. A device according to claim 12, wherein the aperture limiting diaphragm is realized by the limited extension of at least one of the optical elements in the second beam path.

14. A device according to one of the claims 1 to 6, wherein the element limiting the bundle opening, for the light beam bundle entering into the fiber-optic from the second beam path consists of one of the elements of the claims 10 to 13 alone or a combination of these elements.

15. A device according to one of the claims 1 to 14, characterized in that the fiber-optic leading the light to the endoscope consists of a fluid fiber-optic with a high transmission in the fluorescence excitation band.

16. A device according to one of the claims 1 to 14, wherein the fiber-optic consists of a fiber or a fiber bundle with a high transmission in the fluorescence excitation band.

17. A device according to one of the claims 15 and 16, wherein the fiber-optic runs up to the distal end of the endoscope.

18. A device according to one of the claims 1 to 6, wherein the second illumination means is a laser.

19. A device according to one of the claims 1 to 6, wherein the second illumination means is a mixed-gas lamp.

20. A device according to one of the claims 1 to 6, wherein the second illumination means is a light diode or an array of light diodes.

21. A device according to one of the claims 1 to 6, wherein the second illumination means is a short arc lamp.

22. A device according to one of the claims 1 to 6, wherein the second illumination means is a filament lamp.
